(19) United States
(12) Patent Application Publication
(10) Pub. No.: US 2025/0065334 A1
(43) Pub. Date: Feb. 27, 2025

(54) VESSELS, DEVICES, PROCESSES, AND SYSTEMS FOR TRACKING AND MANAGING ITEMS WITH CIRCUITOUS CUSTODY CHAINS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventor: Noah Fichter, New York, NY (US)

(21) Appl. No.: 18/725,042

(22) PCT Filed: Dec. 28, 2022
(86) PCT No.: PCT/US2022/054203

§ 371 Date: Jun. 27, 2024

Related U.S. Application Data
(60) Provisional application No. 63/294,758, filed on Dec. 29, 2021.

(51) Int. Cl. B01L 3/00; B01L 7/00; B65D 25/02; B65D 79/02; B65D 81/18

(52) U.S. Cl. CPC B01L 3/50851; B01L 7/50; B65D 25/02; B65D 79/02; B65D 81/18; B01L 2200/185; B01L 2300/022; B01L 2300/0636; B01L 2300/1894

(57) ABSTRACT

The approach disclosed herein can provide a closed-loop physical supply chain using a precision vessel. An example vessel includes an outer shell and an inner chamber that fits therein. The inner chamber includes a first compartment to removably house a product, and a second compartment with environmental control mechanisms. An enclosed layer, which may be secured to or integrated with the inner chamber, includes a controller and a communication interface to communicate with devices external to the precision vessel. Multiple sensors that monitor the first compartment send signals to the controller of the enclosed layer. A locking mechanism, when engaged, secures the outer shell to the inner chamber, and when disengaged, unlocks the outer shell from the inner chamber to thereby provide access to the first compartment and the product therein. The vessel can track its conditions and locations, and requires verification prior to disengaging the locking mechanism.

### REFERENCE TO RELATED APPLICATIONS

This application claims priority to U.S. Provisional Patent Application No. 63/294,758 filed Dec. 29, 2021, the entirety of which is herein incorporated by reference.

### BACKGROUND

Cell and Gene Therapy (CGT) is a new frontier in medicine. It is science defined by extracting cells, proteins, or genetic materials from a patient or donor, altering them, then reinjecting them back into the patient. Of significant promise are CAR-T treatments, a form of immunotherapy that uses patient T cells to fight cancer, offering patients individualized treatments beyond traditional chemotherapy, surgery, and radiation. Patient T cells, the cells responsible for immune system responses, are separated from a patient's blood via apheresis and are modified in a lab by adding synthetic receptors called chimeric antigen receptors or CAR. The combined CAR-T cells are then expanded into the hundreds of millions and ultimately infused back into the patient where they will further multiply as they recognize and kill cancer cells.

Apheresis, for example, separates and extracts patients' white blood cells from the rest of their blood over the course of a 2-to-3-hour procedure. The white blood cells are then sent off to a laboratory for testing and clinical review of CAR-T requirements. The patient specimen (i.e., the bag of white blood cells) should be properly managed according to each treatment's guideline, most requiring cryopreservation. The preparation, validation, packaging, order placement, shipping and receiving, scheduling, and testing are managed by hospital administrators and clinical staff. The manufacturing process (i.e., adding the CAR and growing the cells) is managed by pharmaceutical companies, the specific company, and requirements dependent upon the treatment.

Clinicians bear the burden of managing the extensive operational processes that start from the point of patient blood collection through to infusion back into the patient. Procedures span pharmaceutical companies, blood banks, laboratories, and medical centers, each with their own requirements and processes. Coordinating the continuum of care requires virtually every organizational level and functional role within the hospital setting. Current management practices consist of paper-based data collection transcribed to spreadsheets, emails, and disparate software solutions.

By 2025, the FDA anticipates “10-20 cell and gene therapy products will be approved annually” (FDA Active Product Development Guidance (January 2019)). CAR-T cycle times (extraction to infusion) can take several weeks to upwards of a couple months depending on market conditions, leaving patients and clinical stakeholders uncertain of the treatment status. As volumes grow and new treatments come to market, the risk of poor quality, fraud, and higher costs are likely to follow. These individualized treatments are costly, with many CAR-T therapies ranging from $300,000 to $500,000 per round of treatment. Insurance claims are heavily scrutinized, and reimbursements are often contested, increasing financial risk to hospital cash flows. Underpinning many issues is the lack of transparency. Tracking status and planning downstream dependencies requires detailed orchestration between hospital administration and clinical operations.

### SUMMARY

A first aspect relates to a precision vessel. In various embodiments, the precision vessel can comprise an outer shell and an inner chamber that fits in the outer shell, the inner chamber comprising a plurality of compartments. The plurality of components can comprise a first compartment to removably house a product. The plurality of components can comprise a second compartment comprising one or more environmental control mechanisms. The precision vessel can comprise an enclosed layer. The enclosed layer can comprise a controller and one or more communication interfaces to communicate with one or more devices external to the precision vessel. The precision vessel can comprise a plurality of sensors to monitor the first compartment. The plurality of sensors can be coupled to the controller of the enclosed layer. The precision vessel can comprise a locking mechanism. The locking mechanism can engage to secure the outer shell to the inner chamber. The locking mechanism can be disengaged to unlock the outer shell from the inner chamber to thereby provide access to the first compartment. The locking mechanism may engage or disengage according to instructions from the controller.

In various embodiments, the one or more environmental control mechanisms of the second compartment can comprise a cooling mechanism. In various embodiments, the cooling mechanism can comprise a refrigeration mechanism that adjusts refrigeration according to signals from the controller. In various embodiments, the cooling mechanism can comprise a cold pack. In various embodiments, the enclosed layer can be secured to the inner chamber. In various embodiments, the enclosed layer can comprise one or more openings for wiring from the plurality of sensors to enter the enclosed layer. In various embodiments, precision vessel can further comprise an output device. In various embodiments, the controller generates an audiovisual indicator to indicate a fault with the precision vessel upon detection of a measurement at or above a threshold. In various embodiments, the audiovisual indicator can be an audible alarm and/or a visible light. In various embodiments, a fault can indicate a temperature that is too high, humidity levels that are too high or too low, forces experienced that are too high (indicating, e.g., a significant collision or fall), and/or the vessel being situated at a location that is not authorized.

In various embodiments, the second compartment can be sealed such that access thereto is restricted when the locking mechanism is disengaged. In various embodiments, the plurality of sensors can comprise a biometric sensor for authentication of a user prior to disengaging the locking mechanism. In various embodiments, the enclosed layer can comprise a memory device. In various embodiments, the controller can record data each time an attempt is made to gain access to the precision vessel. In various embodiments, the plurality of sensors can comprise an accelerometer to detect forces experienced by the precision vessel. In various embodiments, the plurality of sensors can comprise a humidity sensor. In various embodiments, the plurality of sensors can comprise a temperature sensor. In various embodiments, the precision vessel can comprise a locator device. In various embodiments, the locator device can periodically detect a location of the precision vessel. In various embodiments, the locator device can transmit the location to a system via the one or more communication interfaces. In various embodiments, the locator device can be, or can include, a global positioning system (GPS) device. In various embodiments, the precision device can include an accelerometer. In various embodiments, the controller can use the locator device to detect a location of the precision vessel each time a force exceeding a threshold is detected using the accelerometer.

Another aspect relates to a method of using a precision vessel. In various embodiments, the precision vessel comprises: an outer shell and an inner chamber that fits in the outer shell, the inner chamber comprising a plurality of compartments, the plurality of components comprising a first compartment and a second compartment comprising one or more environmental control mechanisms; an enclosed layer comprising a controller and one or more communication interfaces to communicate with one or more devices external to the precision vessel; a plurality of sensors to monitor the first compartment, the plurality of sensors coupled to the controller of the enclosed layer; and a locking mechanism that engages to secure the outer shell to the inner chamber and that disengages to release the outer shell from the inner chamber. The method can comprise placing a product into the first compartment at a first location. The method can comprise fitting the outer shell over the inner chamber. The method can comprise engaging the locking mechanism to secure the outer shell to the inner chamber. The method can comprise providing the precision vessel for shipping to a second location.

In various embodiments, engaging the locking mechanism comprises using an application executing on a user device in communication with the precision vessel. In various embodiments, the product is a sample. In various embodiments, the method can comprise collecting the sample from a subject. In various embodiments, the product is a first product. In various embodiments, the precision vessel is a first precision vessel. In various embodiments, the method comprises removing, from the first precision vessel or a second precision vessel, a second product. In various embodiments, the method comprises administering the second product to a subject. In various embodiments, the second product comprises a modification or derivation of the first product placed into the first compartment of the first precision vessel.

The foregoing general description and following description of the drawings and detailed description are exemplary and explanatory and are intended to provide further explanation of the invention as claimed. Other objects, advantages, and novel features will be readily apparent to those skilled in the art from the following brief description of the drawings and detailed description.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a block diagram illustrating a general architecture for a computer system that may be employed to implement elements of the systems and methods described and illustrated herein, according to various potential implementations.

FIG. 2A is a diagram illustrating a process in which a product is obtained from a source (used interchangeably with “origin” or starting point), is shipped to a location (e.g., an intermediary or intermediate location between a source and a destination) for modification or other handling, and is transported back for receipt by a destination that is the same source, according to various potential implementations.

FIG. 2B is a diagram illustrating potential custody chains involving a source, destination, and one or more intermediary locations, according to various potential implementations.

FIG. 3 is a diagram illustrating an example vessel (along with various sensors) in which the product and the modified product may transported and monitored along the chain of FIG. 2A or 2B, according to various potential implementations.

FIG. 4 depicts an outer shell of a precision vessel, according to various potential implementations.

FIG. 5A depicts an inner chamber of a precision vessel from a first perspective, according to various potential implementations.

FIG. 5B depicts an inner chamber of a precision vessel from a second perspective, according to various potential implementations.

FIG. 6 depicts potential dimensions of an example outer shell, according to various potential implementations.

FIG. 7 depicts potential dimensions of an example inner chamber, according to various potential implementations.

FIG. 8A depicts a front view of an example inner chamber and an enclosed layer of the example inner chamber, according to various potential implementations.

FIG. 8B depicts a top view of the example inner chamber of FIG. 8A and features of an inside of the enclosed layer, according to various potential implementations.

### DETAILED DESCRIPTION

Various embodiments of the disclosure relate to vessels, devices, processes, and systems for tracking and managing items, products, and/or packages that may, but need not, have circuitous custody chains (e.g., a package that has an origin or source that is the same as the package's destination). Certain products or items may be both the input and the output of a circuitous exchange of custody between various organizations and individuals. Each step may require an exchange of information and/or physical action to manage “change of custody,” “chain of identity,” and/or “change of form.” Various embodiments provide a closed-loop physical supply chain with a mirrored digital supply chain. Although the disclosure provides specific examples related to pharmaceuticals in illustrative embodiments of the technology, the technology is not so limited to such use cases, and can be equally useful in other situations in which any item, product, or package is to be more securely and controllably handled and transported, especially if the item, product, or package has one or more intermediary sites (“stops”) moving from an origination site to a destination site.

The various concepts introduced above and discussed in greater detail below may be implemented in any of numerous ways, as the described concepts are not limited to any particular manner of implementation. Examples of specific implementations and applications are provided primarily for illustrative purposes.

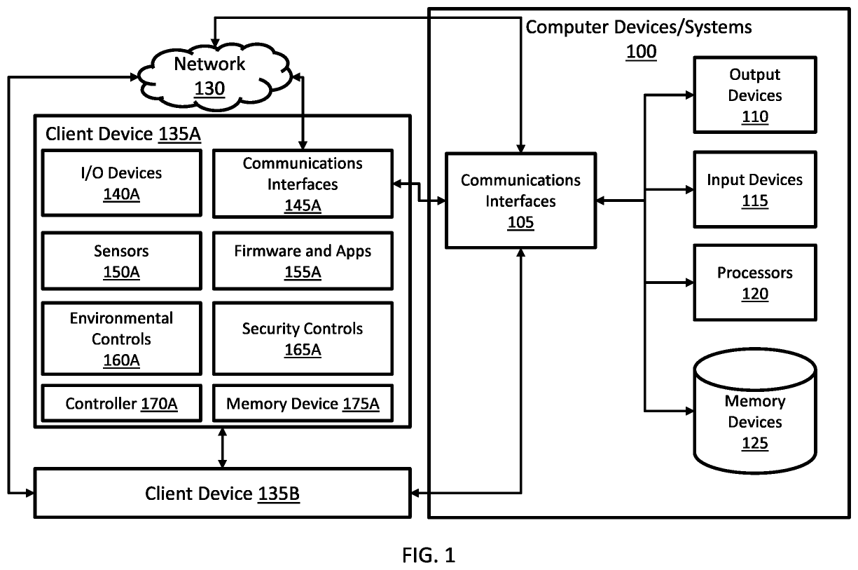

FIG. 1 shows the general architecture of illustrative computer devices/computer systems 100, one or more of which could be employed to implement each of the computer systems discussed herein in accordance with some implementations, as well as client devices 135A and 135B. The computer devices/computer systems 100 can be used to provide information via the network 130 for display. The computer devices/computer systems 100 of FIG. 1 comprise one or more processors 120 communicatively coupled to one or more memories 125, one or more communications interfaces 105, one or more output devices 110 (e.g., one or more display units, speakers, light sources, haptic feedback devices, etc.), and one or more input devices 115 (e.g., microphones, touchscreens, and/or sensors).

In the computer devices/computer systems 100 of FIG. 1, the memory or memories 125 may comprise any computer-readable storage media, and may store computer instructions such as processor-executable instructions for implementing the various functionalities described herein for respective systems, as well as any data relating thereto, generated thereby, or received via the communications interface(s) or input device(s) (if present). The memory 125 can include one or more databases. The processor(s) 120 shown in FIG. 1 may be used to execute instructions stored in the memory or memories 125 and, in so doing, also may read from or write to the memory various information processed and or generated pursuant to execution of the instructions.

The processor(s) 120 of the computer devices or computer systems 100 shown in FIG. 1 also may be communicatively coupled to or made to control the communications interface(s) 105 to transmit or receive various information (data, control signals, executable code or other instructions, etc.) pursuant to execution of instructions. For example, the communications interface(s) 105 may be coupled to a wired or wireless network, bus, or other communication means and may therefore allow the computer devices or computer systems 100 to transmit information to or receive information from other devices (e.g., other computer systems). The communications interface(s) 105 may employ one or more communication protocols such as “Wi-Fi,” “Bluetooth,” radio-frequency identification (RFID), near-field communications (NFC), cellular communications (e.g., 3G, 4G, and/or 5G), etc. While not shown explicitly in the system of FIG. 1, one or more communications interfaces 105 facilitate information flow between the components of the computer devices or computer systems 100. In some implementations, the communications interface(s) 105 may be configured (e.g., via various hardware components or software components) to provide a website as an access portal to at least some aspects of the computer devices or computer systems 100. Examples of communications interfaces 105 may include user interfaces (e.g., webpages), through which users can communicate with computer devices or computer systems 100.

The output device(s) 110 of the computer devices or computer systems 100 shown in FIG. 1 may be provided, for example, to allow various information to be viewed or otherwise perceived in connection with execution of the instructions. Example output devices include display screens (e.g., touchscreen displays), speakers, light sources, haptic devices, and so forth. The input device(s) 115 may be provided, for example, to allow a user to make manual adjustments, make selections, enter data, or interact in any of a variety of manners with the processor during execution of the instructions. Example input devices include cameras, light sensors, touch interfaces (e.g., a touchscreen displays), microphones, computer mice, keyboards, motion sensors, biometric sensors (e.g., fingerprint reader, facial detection device, etc.) and so forth. Additional information relating to a general computer system architecture that may be employed for various systems discussed herein is provided further herein.

Client devices 135A and 135B may be, or may comprise, any combination of user computing devices (e.g., smartphones, tablets, laptops, desktop computers, internet-of-things (IoT) devices, or other smart devices), smart packages, smart containers, global positioning system (GPS) devices, handheld scanners, location detectors, etc. For example, the vessels disclosed herein can be deemed a client device. Client device 135A includes input/output (I/O) devices 140A, such as microphones, cameras, touchscreens; communications interfaces 145A that enable communications through various networks (e.g., the internet) and using various communication protocols (e.g., “Wi-Fi,” “Bluetooth,” cellular, etc.); sensors 150A, such as location and orientation sensors (e.g., GPS devices that detect GPS signals identifying latitude and longitude coordinates associated with locations of client devices, accelerometers, gyroscopes, etc.), ambient sensors (used interchangeably with “environmental sensors”) capable of measuring or detecting ambient conditions such as temperature, humidity, pressure, etc. internal or external to the client device 135A, biometric sensors (e.g., fingerprint scanner, retina scanner, etc.), chemical sensors that detect, for example, certain molecules; and/or shock sensors (e.g., using data from accelerometers); firmware and applications (such as client software applications) 155A which provide various functionality (e.g., hardware control, user interfaces, etc.) with respect to the other components of client device 135A; environmental controls 160A, such as mechanisms for maintaining conditions of the device, such as temperature controls (e.g., heating and/or cooling mechanisms), humidity controls, physical stability controls (e.g., shock absorption), sterilization controls (e.g., emitting ultraviolet or light at other wavelengths to prohibit or otherwise control microorganism growth in different parts of a vessel or for other purposes), etc.; and security controls 165A, such as locking and unlocking mechanisms which may be activated or deactivated via one or more of the other components of client devices 135 (e.g., I/O devices 140A, communications interfaces 145A, biometric sensors 150A, and/or client applications 155A). In some implementations, a security mechanism may be triggered based on a failure of environmental controls. Though not pictured, client device 135B can be equivalently or similarly equipped as client device 135, can include a subset of the components of client device 135A, or can include one or more of the same or similar components as, and/or one or more different components from, client device 135A.

In various embodiments, client device 135A (e.g., a precision vessel as discussed herein) includes a controller 170A that is able to send and/or receive data, directly or indirectly, to/from the other components of client device 135A. For example, in various embodiments, controller 170A can receive data such as inputs from or via I/O devices 140A, status information or other data from or via communications interfaces 145A, measurements or readings from or via sensors 150A, and access attempts from or via security controls 165A. In various embodiments, controller 170A can send data (e.g., commands or other instructions for actions to take) to I/O devices 140A (e.g., messages or information to be conveyed to a user), to communications interfaces 145A (e.g., signals to transmitted to other systems or devices), to sensors 150A (e.g., commands to begin, change, or cease detecting various parameters, such as to begin reading biometric data), to environmental controls 160A (e.g., to engage, change, or disengage various components to change internal or external conditions based on various inputs such as data from sensors 150A), and/or to security controls 165A (e.g., to engage or disengage a locking mechanism based on various inputs such as data from sensors 150A).

Controller 170A can include one or more processors. In various embodiments, controller 170A can include one or more memory devices (e.g., volatile or non-volatile memory devices) that store computer instructions such as processor-executable instructions for implementing the various functionalities described herein for respective devices 135A, as well as any data relating thereto, generated thereby, or received via the communications interface(s) 145A or input device(s) 140A (if present). The processors of controller 170A may execute instructions stored in the memory devices and, in so doing, also may read from or write to the memory devices various information processed and or generated pursuant to execution of the instructions. In various embodiments, controller 170A can be, or can include, a microcontroller board.

Client device 135A can include a memory device 175A, such as a solid state drive (SSD), for (longer-term) storage of data related to the client device 135, such as data on the states, statuses, and locations of device 135A over time, and access thereto. Although not depicted in FIG. 1, device 135A can include one or more batteries to power the various components. The one or more batteries can include separate batteries for various components, one central battery to power various components, and/or one or more backup batteries in case of failure of the first battery. The batteries may be rechargeable through a charging port of client device 135A.

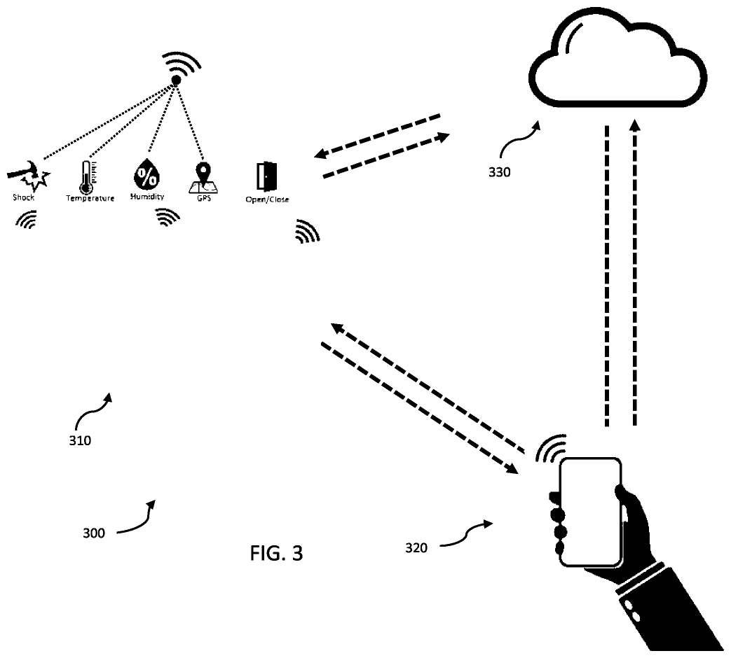

Example computer devices or computer systems 100 include, for example, one or more computer devices or computer systems of logistics service providers, shipping service providers, electronic medical records (EMR) providers, hospitals or other healthcare service providers, pharmaceutical companies, etc. Example client devices 135A and/or 135B may be user computing devices used by, for example: entities (e.g., healthcare providers) involved in initially obtaining a product (e.g., a biological sample); persons involved in transporting or otherwise handling a product along the chain of custody; individuals involved in processing a product being transported; other users associated with the logistics service provider, shipping service provider, EMR provider, healthcare service provider, etc.; and/or a precision vessel (see, e.g., FIG. 3). In various embodiments, computer devices or computer systems 100 and one or more client devices 135A, 135B may be systems and devices of one entity, or of multiple entities.

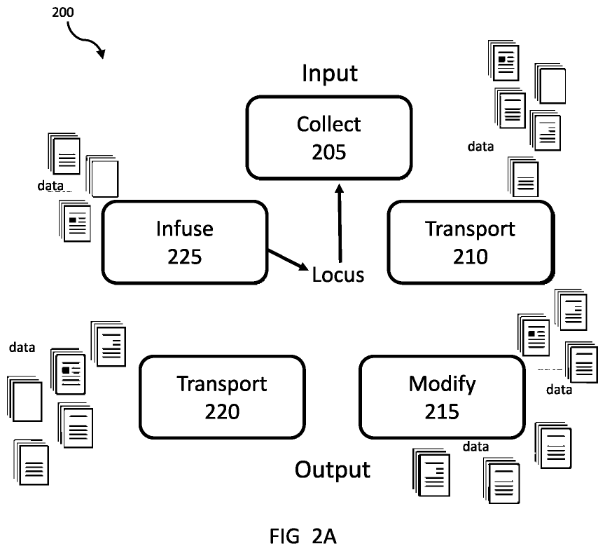

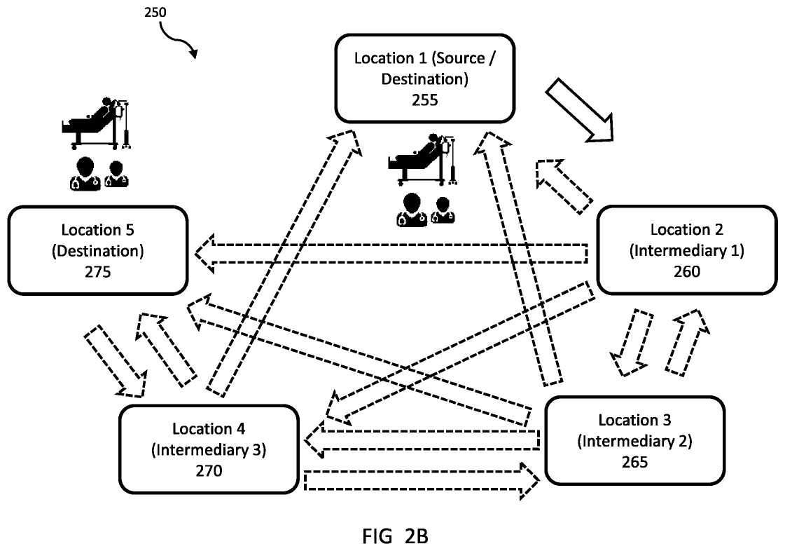

FIG. 2A is a diagram illustrating a non-limiting example of a circuitous process 200, according to various potential embodiments. An item, sample, or product (collectively referred to as “product” for convenience) that is a first product may be collected or otherwise obtained (205) from a source (e.g., collected from an individual or other “locus”). The first product may be transported (210) to an intermediary location (e.g., a pharmaceutical company, manufacturing facility, etc.) for modification or other handling (215), or for derivation of a second product based on the first product. The first product (or the second product derived from the first product) may be transported (220) back again for receipt (of the modified first product or the newly-derived second product) by the same source (e.g., the same individual or other locus), according to various illustrative potential implementations. Once the modified or derived product reaches the locus at the source, it may be provided to the locus (e.g., injected into an individual), with or without further modification. At each step, data (e.g., documents, sensor readings, a record of locations and actions taken, quality check information, etc.) is generated and/or modified.

Referring to FIG. 2B, it is noted that the source and destination need not be the same, and the route taken can vary greatly. For example, a first individual may deposit or otherwise provide a product at a first location 255 (e.g., a first clinic or other collection site), which may serve as the origin or source. The product, which is in a first state when first obtained at first location 255 (e.g., the origin), may optionally be modified to be in a second state while still at the first location 255. The product (in the first state or the second state) may be secured or otherwise situated (e.g., by the first individual, who may be a patient, or by a second individual, who may be a healthcare provider or other professional) in a precision vessel and the vessel sealed. The vessel with the product may be transported to a second location 260 (e.g., a first intermediary location). At the second location 260, the product may be modified to a third state. Optionally, the vessel may also be transported to a third location 265 (e.g., a second intermediary location) at which it may be further modified to a fourth state, transported to a fourth location 270 (e.g., a third intermediary location) at which it may be further modified to a fifth state, and so forth. From the final intermediary location (e.g., the fourth location 270 which may be the third intermediary location), the product (e.g., in a fifth state) may be transported to a fifth location 275 (e.g., its final destination). The fifth location 275 may be the same as the first location 255, or may be different from the first location 255. For example, the final destination may be a second clinic, the first individual's home, etc., where the product may be removed from the vessel (by the first individual who provided the product, by the second individual, or by another individual such as another healthcare provider) for use or handling.

The distance between locations may vary. For example, two locations may be at the same premises (such that, e.g., an intermediary location is “on the premises” with respect to a prior location), or a subsequent location may be in another part of a city, in another city, in another state, in another country, or in another continent with respect to a prior location. At each location, data (e.g., documents, sensor readings, a record of locations and actions taken, quality check information, etc.) is generated and/or modified. Data can be stored in the memory of the vessel (e.g., a solid state drive) and/or transmitted to external devices and/or systems that are local to, or remote from, the vessel. In some embodiments, data can be stored on a distributed ledger in the interest of enhanced security and/or transparency.

Modifications to the state of a sample or other contents may result from the addition of materials to, subtraction or removal of materials from, and/or change to materials in a vessel (e.g., conversion of materials from one state to another state). In some embodiments, modifications to the product may not require removal of the product from the vessel. State-changing modification of the product may comprise addition of various materials (e.g., certain reactants or other agents) through an opening in the vessel, or into a chamber of the vessel. The modification may comprise application of electrical and/or magnetic fields, emission of lights of particular wavelengths, treatment with radiation sources, storage at particular environmental conditions (e.g., certain temperatures and/or humidity levels), etc. It is noted that, “modification” of a product, as used herein, could refer to a change in state (e.g., addition, subtraction, or conversion of compositions, agents, components, etc.) of the product, and/or to a generation of a second product that is derived from the original product (e.g., created based on the contents of the original product).

FIG. 3 depicts an example system 300 illustrating the interface between one or more example vessels 310 (e.g., client device 135A), one or more example user devices 320 (e.g., client devices 135B), and other devices or systems (e.g., computer devices/systems 100) that may be accessed via any of a variety of communications networks 330. The precision vessel 310 could be used to transport a product from a source to an intermediary location at which the product may be processed or otherwise modified, and to transport the modified product from the intermediary location to the source or to another location. The vessel 310 may include, in various embodiments, a combination of the devices or components disclosed herein. For example, the vessel 310 can include various sensors, such as: sensors that detect shocks to the vessel 310 to, for example, detect drops or collisions (e.g., using one or more accelerometers), sensors that measure temperatures and temperature changes inside and/or outside of the vessel or its contents (e.g., the product or modified product), sensors that detect humidity or moisture inside or outside of the vessel or a container therein, GPS or other location sensors, sensors for gauging the integrity of the vessel or its components (e.g., to identify a potential break in, or other compromise to, the vessel or its contents by detecting, e.g., sounds of a break in plastic or glass, and/or piezoelectric sensors for detecting a dent in, or other change in shape of, the vessel that would result in a detectable force), sensors for detecting when and for how long the vessel (and/or a container therein) has been opened and closed, etc. Other example sensors include orientation sensors (e.g., gyroscopes), atmospheric pressure sensors, proximity sensors to detect objects in its surrounding, cameras and/or microphones to record images, videos, and ambient sounds in the vessel's surroundings or the contents thereof, audio and/or visual inputs and/or outputs (e.g., speakers, light sources, touchscreens or other displays, keypads, etc.) to allow for interaction with users coming into contact with the vessel 310, etc. The components illustrated in FIG. 3 are non-limiting example components that can be incorporated into various embodiments of precision vessel 310, but any combination of components disclosed herein can be incorporated into other potential embodiments of vessel 310.

The vessel 310 can communicate with one or more devices 320. Non-limiting examples of potential devices 320 include mobile or non-mobile devices, such as smartphones, tablets, laptops, desktop computers, workstations, sensors, communications routers, mainframes, or other systems or devices. Devices 320 with which vessel 310 can communicate can also include one or more other precision vessels 310.

Devices 320 can be communicated with directly, wirelessly or through wires, and would be located within a maximum range of the wireless or wired communication protocol being implemented. Communications between vessel 310 and devices 320 can employ one or more wireless or wired communication protocols, such as Wi-Fi, “Bluetooth” (e.g., “Bluetooth LE” for low-power devices), “Zigbee” (e.g., IEEE 802.15.4-based specification), LoRa (e.g., “long range” communications), “NBIoT” (Narrow Band Internet of Things), Thread (e.g., an IPv6-based, mesh networking technology for IoT products), near-field communication (NFC), I2C (Inter-integrated Circuit bus), SPI (Serial Peripheral Interface bus), Ethernet and/or other local area networks (LANs), RS-232 (Recommended Standard 232), RS-485, UART (Universal Asynchronous Receiver Transmitter), USB (Universal Serial Bus, such as USB 1.1 employing USB-A and/or USB-B connectors, USB 2.0 employing USB-A, USB-B USB Micro A, USB Micro B, USB Mini A, USB Mini B, and/or USB-C connectors, and/or USB 3.2 employing USB-A, USB-B, USB Micro B, and/or USB-C connectors).

Vessels 310 can also communicate with other devices and systems through other networks 330 (illustrated as “the cloud”), such as non-local devices and systems of other entities accessed via an Internet-based network. Communications with such other devices can be through cellular networks (e.g., 3G, 4G, 5G) or through other communications mechanisms (e.g., Wi-Fi). The other entities can be affiliated networks (e.g., or the same entity with shared security protocols) or unaffiliated networks (e.g., of different entities employing different security protocols). In non-limiting examples, vessel 310 can communicate, via networks 330, with devices or systems of any entity associated with any terminal location (e.g., source or destination) or any intermediary location (e.g., clinics, pharmacies, etc.).

The communications can be for authentication (e.g., to activate or deactivate a locking mechanism), verification (e.g., to confirm that a certain action with respect to the vessel is permitted, to confirm a location is permitted, etc.), tracking (e.g., to keep a record of locations of the vessel, ambient conditions surrounding the vessel, internal conditions or contents of the vessel, etc., with time data indicating when and for how long the vessel remained in certain locations or environmental conditions or certain contents were in the vessel), recording (e.g., what actions were taken, when, and by whom for the vessel at various locations), and/or updates (e.g., changes in what actions are to be taken, information that is to be provided to, or collected from, entities or individuals interacting with the vessel, minimum and/or maximum internal and/or external environmental conditions that trigger alarm conditions, and/or firmware updates, etc.).

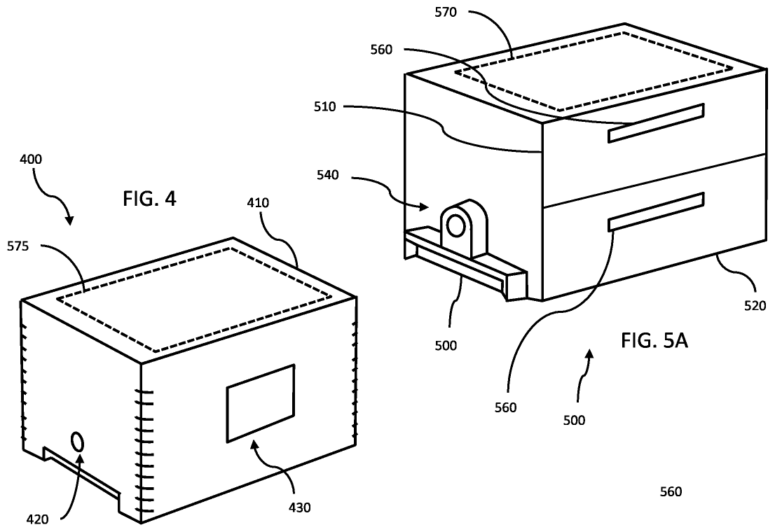

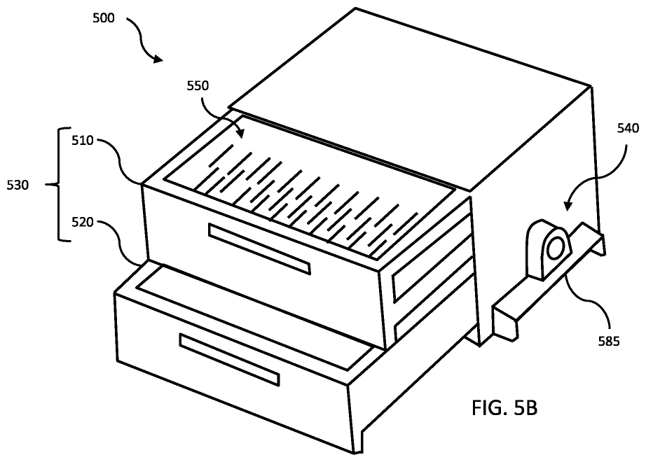

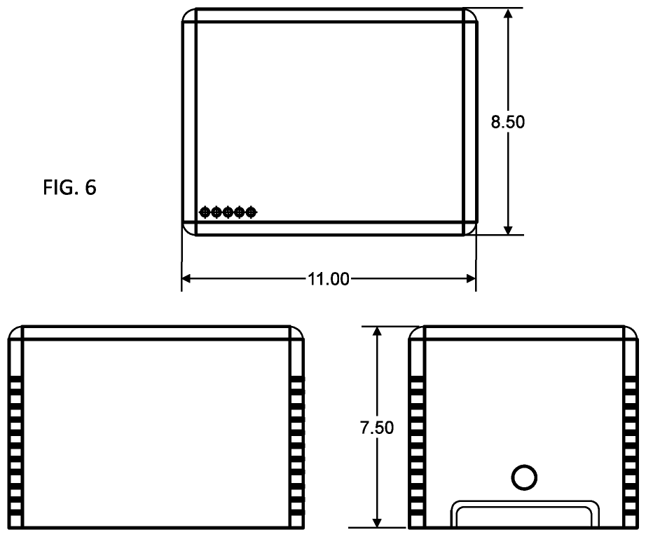

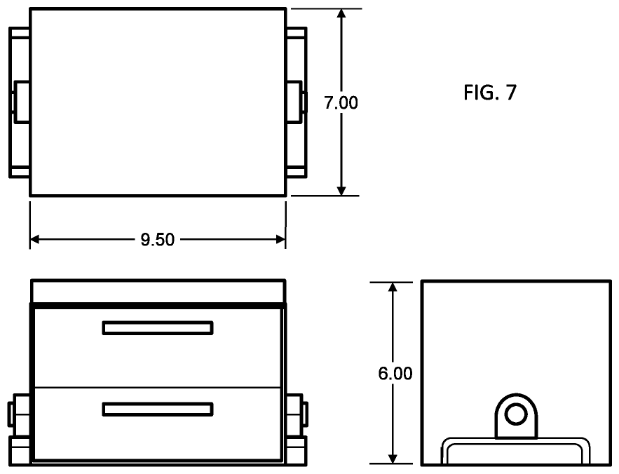

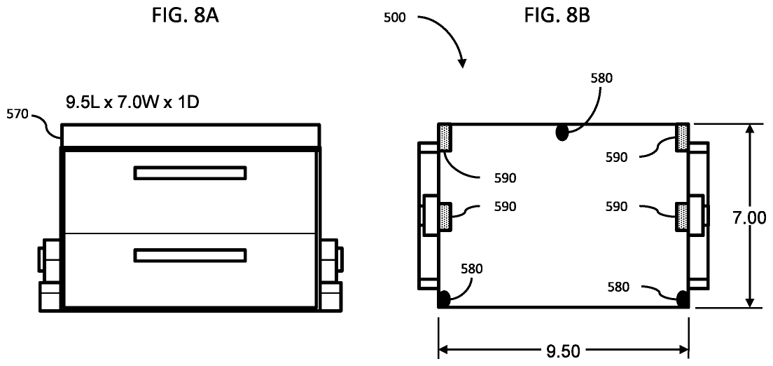

FIGS. 4, 5A, and 5B depict components of an example precision vessel (e.g., vessel 310). The vessel 310 may include any of the components discussed with respect to client device 135A. In various embodiments, the vessel may be a “Precision Medicine Vessel” (PMV) that is an IoT-enabled container that physically stores and controls access to products (e.g., patient specimens), collects and outputs information asynchronously about the specimen, and records interactions with the product. Here, being IoT-enabled indicates that, in various embodiments, the vessel is equipped with sensors (e.g., sensors 150A), processing capability (e.g., controller 170A), and the ability to connect and exchange data (e.g., via communications interfaces 145A) with other devices and systems over the Internet or via other communication protocols (e.g., RFID, NFC, and/or “Bluetooth”).

The example vessel physically comprises and outer shell 400, an inner chamber 500 with one or more compartments 530, such as drawers 510 and 520, and the locking mechanism 530 that secures these physical components together as one unit. The outer shell 400 may be five-sided (e.g., having a cubicle structure without one of six side walls, such as being without a bottom or without a side wall), such that it could be slipped over the inner chamber 500 (for securing the outer shell 400 with the inner chamber 500), and slid off the inner chamber 500 (for removal or separation from the inner chamber 500).

Outer shell 400 may be insulated to reduce heat transfer into or out of the vessel 310. In certain embodiments, outer shell 400 may also be insulated to reduce electromagnetic radiation permeating into the vessel 310, such as light that could affect the contents of the vessel (e.g., if the vessel 310 is housing photoactive components) and/or add heat and thereby increase the temperature inside the vessel 310. The 5-sided outer shell can fit over the inner chamber of drawers and lock into place to form an insulated vessel. The outer shell 400 may include a cutout 420 sized and shaped (e.g., being circular) to receive or otherwise accommodate a protrusion of the locking mechanism 530. The protrusion extends through the cutout 420 and, when the locking mechanism is engaged, restricts removal of the inner chamber from the outer shell, and when the locking mechanism is disengaged, does not restrict removal of the inner chamber from the outer shell.

The outer shell 400 may also include one or more interfaces 430 (e.g., I/O devices 140A and/or sensors 150A) for interaction with a user. The interfaces 430 may include, for example, any combination of one or more of: displays and/or visual indicators (e.g., LCD screen, touchscreen display, and/or LED lights); audio sources (e.g., a speaker for providing verbal or other feedback to a user, an alarm, and/or other sounds); a keypad (e.g., with alphanumeric characters for entry of security codes, etc.); biometric scanners (e.g., a fingerprint reader or facial scanner); and/or input devices (e.g., microphones, cameras or other optical readers for detecting surroundings and/or for recording audiovisual images and/or videos, etc.). The walls of the outer shell 400 can be made of high-density polyethylene and filled with insulating polyurethane foam to maintain temperature.

In various embodiments, inner chamber 500 is a chamber with multiple parallel, horizontal drawers stacked one above another that slide in and out to access contents. The drawers can range in size, quantity, and material to fit within the inner chamber and to accommodate, for example, patient specimen(s), labeling and packaging materials, cooling mechanisms (e.g., cold pack, refrigerant, dry ice, etc.), environmental requirements (e.g., vacuum insulated panels for thermal insulation of cryopreserved specimens), etc.

Inner chamber 500 may include a plurality of drawers 530, such as a drawer 510 to hold a product (e.g., a patient sample), and a drawer 520 to house various components to interact with or otherwise affect the contents of drawer 510. In various embodiments, drawer 510 can include, for example, various components that regulate, monitor, and/or safeguard the contents of drawer 510. For example, drawer 510 can include any combination of one or more of: a shock absorbing mechanism (e.g., grips that secure a sample vial with springs that dampen forces experienced by the sample vial); a heat sink; and/or sensors for detecting conditions of drawer 510 (e.g., sensors for temperature, humidity, amount of light crossing into drawer 510, etc.). Drawer 510 can include slits 550 (or any other openings) to facilitate, for example, heat exchange between drawer 510 and drawer 520 and/or to otherwise allow better access to drawer 510 from drawer 520.

In various embodiments, the drawer 520 could house components that can regulate, monitor, and/or safeguard the contents of drawer 510. For example, drawer 520 can include any combination of one or more of: cooling components (e.g., a cold pack, a heat sink, a refrigeration mechanism, and/or one or more fans) to reduce the temperature of the contents of drawer 510 down; a heat source (e.g., a heat lamp, metallic wires connected to a battery used to generate heat, etc.) to increase the temperature of the contents of drawer 510; a light source (e.g., to emit light at particular wavelengths that might provide antimicrobial or other effects in drawer 510); an electromagnet (e.g., a magnet that could be activated upon application of electrical energy in varying patterns to apply a magnetic field to the contents of the drawer 510 to, e.g., attract, repel, or otherwise manipulate magnetic beads or other magnetic elements in a liquid to cause mixing of samples or other components of the drawer 510); and/or a humidifier and/or a dehumidifier to change the control the humidity of the contents of drawer 510. Each drawer 530 can include a handle 560 to facilitate the respective drawer from being pulled out from the inner chamber 500. The handles 560 may be, for example, protrusions or cutouts that could be more easily gripped to pull the drawer open. In various embodiments, an integrated handle 585 on the bottom of the inner chamber ensures the outer shell and inner chamber fit securely together and can be picked up without decoupling when unlocked.

Also referring to FIG. 9B, in various embodiments, an enclosed layer 570 can comprise the top portion of the inner chamber, providing space for a controller (e.g., a microcontroller board), “Bluetooth” module (and/or other communication interfaces), solid state drive (SSD) (and/or other memory devices), one or more batteries, and one or more sensor circuit connections with sensors of the vessel. Electrical components may be enclosed in, for example, the top one-inch (or other dimensions as suitable to fit the components) of the inner chamber 500. The enclosed layer 570 may be sealed by, for example, a 0.25-inch panel. The panel may be sealed or otherwise secured with one or more fastening mechanisms 580 (such as adhesive, 0.5-inch screws, or other fasteners) at, for example, two corners and a center of an opposing side. The perimeter of the enclosed layer 570 can include slits 590 (e.g., drilled slits or other openings) situated at, for example, the back two corners and at the midpoints of the left and right widths to accommodate sensor wires extending into the enclosed layer 570 from elsewhere in the vessel (e.g., from one or more drawers 530). In certain embodiments, the enclosed layer 570 can be placed at another portion of the inner chamber 500. In other embodiments, an enclosed layer 575 may instead be incorporated with the outer shell 400. In such embodiments, a controller in the enclosed layer 570 would communicate wirelessly or through a wire extending from the outer shell 400 to the inner chamber 500. In certain embodiments, the enclosed layer 575 of the outer shell 400 can be in addition to the enclosed layer 570 of the inner chamber 500, with one or more of the same components as the enclosed layer 570 so as to, for example, provide redundancy in case one of the components fails, or one or more different components as deemed suitable in light of considerations such as reducing potential sources of failure, undesirable heating from components in the enclosed layers 570/575, etc.

In various embodiments, the locking mechanism 530 can be operated by a servo motor. The servo motor may enable remote locking and unlocking via, for example, an integrated software application. The servo motor may communicate with a software application on the user's smartphone via a Bluetooth beacon (or other wireless communication) that can be embedded within the inner chamber and connected to the microcontroller board. A user's smartphone (e.g., client device 135B of FIG. 1 and/or device 320 of FIG. 4) facilitates two-way communication between the vessel and the cloud computing environment (e.g., one or more computer devices/systems 100 accessed via networks 130) in which credentialing is performed. Access is granted or denied based upon authorization and authentication requirements executed by the software application, sending back the communication to lock or unlock the vessel via the user's smartphone.

As an example workflow regarding the locking mechanism 530, a user may log into an application on device 320, and be authenticated via the cloud. As used herein with respect to various embodiments, “the cloud” refers to one or more computing devices/systems 100 with which the vessel 310 (or other client device 135A) and/or the user device 320 (or other client device 135B) communicates via a network 330. The user device 320 may connect to the vessel 310 via Bluetooth or any other of the communication protocols discussed herein.

In various embodiments, each time a user logs in and connects to the vessel via Bluetooth, USB, or other connection (wired or wireless), all stored data in the memory of the vessel (e.g., the SSD of the PMV) is transmitted to the cloud (e.g., to a central system or device 100 that tracks and monitors vessels). The vessel responds with a unique vessel ID, and the application running on device 320 forwards the unique vessel ID to the cloud. The cloud (e.g., the central system 100) responds with an indication of which actions are authorized for that user with respect to the specific vessel with the unique vessel ID.

In various embodiments, the user may request that the vessel be unlocked (e.g., by selecting an “Open” icon in a graphical user interface or otherwise submitting a request for access to the contents of the vessel via the application). The application then requests a One-Time Password (OTP) challenge from the vessel, which generates a time-based OTP (“TOTP”) challenge (e.g., a challenge question that is only valid for a predetermined amount of time, such as 1 minute, 5 minutes, 30 minutes, or 1 hour) via a software token. The application forwards the challenge to the cloud (e.g., central computing system 100), which generates the OTP based on the challenge. The cloud sends an OTP to the user application, which forwards the OTP via the user's Bluetooth (or other) connection to the vessel. The vessel then executes an unlock command to cause the servo lock to unlatch.

A baseline use case will now be discussed to illustrate aspects and features of various potential embodiments of the disclosed technology. In various embodiments, the vessel is a PMV that serves as a storage medium for patient specimen(s) involved in medical procedures or processes in which the specimen changes custody between individuals and/or organizations. The PMV ensures the specimen exchanging custody was accessed by the appropriate stakeholder(s) and the conditions of the specimen are monitored as it changed custody (e.g., the correct stakeholder(s) under known condition(s).

Various embodiments may employ asynchronous information: data generated by sensors, stored on the PMV's solid state drive, then transmitted via Bluetooth (or other communication protocol) to the user's smartphone and ultimately to a cloud environment for processing. The PMV's microcontroller board can accommodate sensors that generate information related to piezoelectric measurements: pressure, acceleration, temperature, strain, or force; location/GPS; humidity; proximity; rotation; gas or chemical levels; and/or status of locking mechanism (e.g., open/unlocked vs. closed/locked).

In various embodiments, the vessel can record interactions: data generated from physical interactions with the PMV, facilitated by a software application. As used herein, the vessel may employ authentication requests (e.g., individuals are who they says they are) and authorization requests (e.g., an individual is allowed to do what the individual is asking to do) in determining whether and when to lock and/or unlock the locking mechanism to open (allow a user access into one or more parts of the vessel, such as one or more drawers) or close (restrict access to one or more drawers) the vessel.

In certain embodiments, different users (e.g., particular individuals, particular entities, particular devices or systems, etc.) can be granted access to different portions of the vessel, each portion being differently maintained or otherwise differently equipped (e.g., different sensors for different measurements relevant to what is to be stored in that portion, different components for maintaining different environmental conditions, etc., as suitable for what is to be placed in that portion. For example: a first user may be allowed to access a first drawer (but not a second drawer a or third drawer) with a first product (e.g., only if the first user is authenticated and only if the first user and vessel are physically located at a particular location) so the first user can access the first product and replace the first product with a first modified product in the first drawer; a second user may be allowed to access the second drawer (but not the first drawer or the third drawer) with a second product (e.g., only if the second user is authenticated and only if the second user and vessel are physically located at another particular location) so the second user can access the second product and replace the second product with a second modified product in the second drawer; and a third user may be allowed to access both the first drawer and the second drawer (e.g., only if the third user is authenticated and only if the third user and vessel are physically located at yet another particular location) so the third user can access the first modified product and the second modified product and generate a third product derived from the combination of the first modified product and the second modified product. The third product may then be inserted into the first drawer, the second drawer, or a potential third drawer that is differently equipped as suitable for preservation of the third product.

In various embodiments, a modified or derived product can be added to the same vessel from which the original product was accessed, or to another vessel. If the modified or derived product is to be added to the same vessel, in various embodiments, the vessel may be stored in various environmental conditions or in a “standby” mode that preserves energy. In certain embodiments, one or more batteries of the vessel may be rechargeable, so that once the vessel is received, it may be, for example, plugged in or otherwise recharged for the remainder of (or another leg of) its chain of custody. Recharging or other preservation actions may be required for verification or authorization to users to perform certain actions. In certain embodiments, an ancillary drawer (e.g., a drawer with a cold pack) may also be accessible to allow for replacement of a spent component (e.g., to replace a used cold pack with a fresh cold pack).

In various embodiments, chain of custody may be maintained through digitally-captured documentation of the sequential change of custody or control of patient specimen. Components of the PMV can be configured to accommodate, for example, blood (human and/or other animals); bone marrow and stem cells; white and red blood cells (e.g., granulocytes, monocytes, lymphocytes, neutrophils, eosinophils, basophils, macrophages, erythrocytes, platelets); human cells (e.g., plasma, cytoplasm, ribosomes, and DNA); fresh, frozen, or formalin-fixed human tissue; organs and body parts (e.g., to be transported for transplants); ovum/ova, zygotes, and embryos; other bodily fluids (e.g., semen, saliva, urine, bile, mucus, breast milk, interstitial fluid (tissue fluid), chyme, transudate, hemoglobin, pus, and/or cerebrospinal fluid.

Implementations of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. The program instructions can be encoded on an artificially generated propagated signal, e.g., a machine-generated electrical, optical, or electromagnetic signal that is generated to encode information for transmission to suitable a receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can include a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or on data received from other sources.

The terms “data processing apparatus”, “data processing system”, “user device” or “computing device” encompasses all kinds of apparatuses, devices, and machines for processing data, including by way of example a programmable processor, a computer, a system on a chip or multiple chips, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from read only memory or random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), for example. Devices suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube), plasma, or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can include any form of sensory feedback, e.g., visual feedback, auditory feedback, or tactile feedback, and input from the user can be received in any form, including acoustic, speech, or tactile input. In addition, a computer can interact with a user by sending documents to and receiving documents from a device that is used by the user, for example, by sending webpages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front end component, e.g., a client computer having a graphical user interface or a web browser through which a user can interact with an implementation of the subject matter described in this specification, or any combination of one or more such back-end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system such as system 1900 or system 100 can include clients and servers. For example, the content management system 130 can include one or more servers in one or more data centers or server farms. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. The components of content management system 130 may be a single module, a logic device having one or more processing modules, one or more servers, or part of a search engine.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations or implementations.

Embodiments of the apparatuses and processes described above are not intended to be limiting of the devices, apparatuses, systems, or processes described above in any manner. The non-limiting potential embodiments related to an application of the disclosed approach to Cell and Gene Therapy (CGT) in general, involving extracting cells, proteins, or genetic materials from a patient or donor, altering them, then re-injecting them back into the patient, such as CAR-T treatments, a form of immunotherapy that uses patient T cells to fight cancer, offering patients individualized treatments beyond traditional chemotherapy, surgery, and radiation. The disclosed approach is not limited to such applications, however, and instead can be applied to other products, processes, and entities.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about,” “approximately,” or “substantially” will be understood by persons of ordinary skill in the art and will vary to some extent depending upon the context in which it is used. If there are uses of the term which are not clear to persons of ordinary skill in the art, given the context in which it is used, “about,” “approximately,” or “substantially” will mean up to plus or minus 10% of the particular term—for example, “about 10%” would mean “9% to 11%.” It is to be understood that when “about,” “approximately,” or “substantially” precedes a term, the term is to be construed as disclosing “about,” “approximately,” or “substantially” the term as well as the term without modification by “about,” “approximately,” or “substantially”—for example, “about 10%” discloses “9% to 11” as well as discloses “10%.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 items or components refers to groups having 1, 2, or 3 items or components. Similarly, a group having 1-5 items or components refers to groups having 1, 2, 3, 4, or 5 items or components, and so forth.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description, or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. Although the examples provided herein relate to augmented reality experiences, the systems and methods described herein can include applied to other environments. The foregoing implementations are illustrative rather than limiting of the described systems and methods. The scope of the systems and methods described herein is thus indicated by the appended claims, rather than the foregoing description, and changes that come within the meaning and range of equivalency of the claims are embraced therein.

The indefinite articles “a” and “an,” as used herein in the specification and in the claims, unless clearly indicated to the contrary, should be understood to mean “at least one” or “one or more.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed:

1. A precision vessel comprising:
an outer shell and an inner chamber that fits in the outer shell, the inner chamber comprising a plurality of compartments, the plurality of components comprising:
a first compartment to removably house a product; and
a second compartment comprising one or more environmental control mechanisms;
an enclosed layer comprising a controller and one or more communication interfaces to communicate with one or more devices external to the precision vessel;
a plurality of sensors to monitor the first compartment, the plurality of sensors coupled to the controller of the enclosed layer; and
a locking mechanism that, when engaged, secures the outer shell to the inner chamber, and when disengaged, unlocks the outer shell from the inner chamber to thereby provide access to the first compartment.

2. The precision vessel of claim 1, the one or more environmental control mechanisms of the second compartment comprising a cooling mechanism.

3. The precision vessel of claim 2, wherein the cooling mechanism comprises a refrigeration mechanism that adjusts refrigeration according to signals from the controller.

4. The precision vessel of claim 2, wherein the cooling mechanism comprises a cold pack.

5. The precision vessel of claim 1, wherein the enclosed layer is secured to the inner chamber.

6. The precision vessel of claim 1, wherein the enclosed layer comprises one or more openings for wiring from the plurality of sensors to enter the enclosed layer.

7. The precision vessel of claim 1, the precision vessel further comprising an output device, wherein the controller is configured to generate an audiovisual indicator to indicate a fault with the precision vessel upon detection of a measurement at or above a threshold.

8. The precision vessel of claim 1, wherein the second compartment is sealed such that access thereto is restricted when the locking mechanism is disengaged.

9. The precision vessel of claim 1, wherein the plurality of sensors comprises a biometric sensor for authentication of a user prior to disengaging the locking mechanism.

10. The precision vessel of claim 1, wherein the enclosed layer further comprises a memory device, and wherein the controller records data each time an attempt is made to gain access to the precision vessel.

11. The precision vessel of claim 1, wherein the plurality of sensors comprises an accelerometer to detect forces experienced by the precision vessel.

12. The precision vessel of claim 1, wherein the plurality of sensors comprises a humidity sensor.

13. The precision vessel of claim 1, wherein the plurality of sensors comprises a temperature sensor.

14. The precision vessel of claim 1, further comprising a locator device.

15. The precision vessel of claim 14, wherein the locator device periodically detects a location of the precision vessel and transmits the location to a system via the one or more communication interfaces.

16. The precision vessel of claim 14, the precision device further including an accelerometer, wherein the controller uses the locator device to detect a location of the precision vessel each time a force exceeding a threshold is detected using the accelerometer.

17. A method of using a precision vessel,
the precision vessel comprising:
an outer shell and an inner chamber that fits in the outer shell, the inner chamber comprising a plurality of compartments, the plurality of components comprising a first compartment and a second compartment comprising one or more environmental control mechanisms;
an enclosed layer comprising a controller and one or more communication interfaces to communicate with one or more devices external to the precision vessel;
a plurality of sensors to monitor the first compartment, the plurality of sensors coupled to the controller of the enclosed layer; and
a locking mechanism that engages to secure the outer shell to the inner chamber and disengages to unlock the outer shell from the inner chamber and thereby provide access to the first compartment; and
the method comprising:
placing a product into the first compartment at a first location;
fitting the outer shell over the inner chamber; and
engaging the locking mechanism to secure the outer shell to the inner chamber.

18. The method of claim 17, wherein engaging the locking mechanism comprises using an application executing on a user device in communication with the precision vessel.

19. The method of claim 17, wherein the product is a sample, the method further comprising collecting the sample from a subject.

20. The method of claim 17, wherein the product is a first product, and wherein the precision vessel is a first precision vessel, the method further comprising removing, from the first precision vessel or a second precision vessel, a second product and administering the second product to a subject, wherein the second product comprises a modification or derivation of the first product placed into the first compartment of the first precision vessel.
